(19) United States
(12) Patent Application Publication
(10) Pub. No.: US 2010/0198345 A1
(43) Pub. Date: Aug. 5, 2010

(54) CALCIUM PHOSPHATE COATED IMPLANTABLE MEDICAL DEVICES, AND ELECTROPHORETIC DEPOSITION PROCESSES FOR MAKING SAME

(75) Inventors: Mehrdad Keshmiri, Burnaby (CA); Tomasz Troczynski, Vancouver (CA)

Correspondence Address: RISSMAN HENDRICKS & OLIVERIO, LLP, 100 Cambridge Street, Suite 2101, BOSTON, MA, 02114, US

(73) Assignee: The University of British Columbia, Vancouver, BC (CA)

(21) Appl. No.: 12/305,877

(22) PCT Filed: Jun. 19, 2007
(86) PCT No.: PCT/CA2007/001081

§ 371 Date: Mar. 29, 2010

Related U.S. Application Data
(60) Provisional application No. 60/814,897, filed on Jun. 20, 2006.

(51) Int. Cl. A61F 2/06; C25D 13/02

(52) U.S. Cl. 623/1.46; 204/510; 204/509

(57) ABSTRACT

This invention relates to novel calcium phosphate coated implantable medical devices, and electrophoretic deposition processes for making same. A process of coating an implantable medical device with a calcium phosphate coating comprising: (a) pretreating a substrate with an alkaline solution; (b) preparing a slurry comprising a solvent and a defined size range of calcium phosphate particles; (c) immersing the pretreated substrate in the slurry; and (d) coating the calcium phosphate particles onto the pretreated substrate by electrophoretic deposition.

### FIELD OF THE INVENTION

This invention relates to novel calcium phosphate coated implantable medical devices, and electrophoretic deposition processes for making same.

### BACKGROUND OF THE INVENTION

Hydroxyapatite [Ca₁₀(PO₄)₆(OH)₂] (HAP) is a ceramic biomaterial with excellent bioactivity and biocompatibility with living tissue. The chemical composition of HAP is very similar to that of bone apatite. HAP is also a bio-resorbable compound capable of absorbing and binding to a variety of molecules such as proteins, enzymes, and other organic components of body fluids such as blood. Most investigations of HAP have focussed on processing routes, characterization methods, and applications of this material as an enhanced coating for biomedical implants, such as orthopaedic and dental implants. The open pore structure of HAP enables penetration of the bone tissue into such coatings, which leads to a higher mechanical integrity and better osseointegration of the coated implant surfaces with host tissue. Several techniques have been utilized for preparing coatings of HAP and other calcium phosphates. Techniques include biomimetic processes, plasma spraying, sputtering, pulsed laser deposition, polymeric route, sol-gel processing, electrochemical deposition, and electrophoretic deposition.

U.S. Pat. No. 5,171,326 entitled “Calcium Phosphate Ceramics For Bone Tissue Calcification Enhancement” discloses electrophoretic deposition (EPD) coating of oxyhydroxyapatite, and alpha- and beta-tricalcium phosphate, on metal surfaces. Materials and processes for enhancing bone ingrowth in porous surfaces, such as titanium mesh implants are disclosed. A similar patent (U.S. Pat. No. 4,990,163 entitled “Method of Depositing Calcium Phosphate Ceramics for Bone Tissue Calcification Enhancement”) was issued earlier with minor differences.

WO Patent No. 03/039609 entitled “Deposition of Coatings on Substrates” discloses coating a material comprising calcium phosphate by EPD. The methods of deposition of calcium phosphate-based materials are disclosed, in general, through either coprecipitation of ions, or particles.

U.S. Pat. No. 5,258,044 entitled “Electrophoretic Deposition of Calcium Phosphate Material on Implant” discloses the deposition of amorphous calcium phosphate, produced through sol-gel processing in the form of a colloidal water-based mixture, on a metallic implant by EPD. The gel-derived material is then sintered at relatively high temperatures of up to 1350° C.

### SUMMARY OF THE INVENTION

One aspect of the present invention is directed to a process of coating an implantable medical device with a calcium phosphate coating comprising: (a) pretreating a substrate with an alkaline solution; (b) preparing a slurry comprising a solvent and a defined size range of calcium phosphate particles; (c) immersing the pretreated substrate in the slurry; and (d) coating the calcium phosphate particles onto the pretreated substrate by electrophoretic deposition.

Further aspects of the present invention are directed to an implantable medical device, a flexible implantable medical device, a stent, or a cardiovascular stent made by the foregoing process.

### DRAWINGS

Exemplary embodiments are illustrated in referenced figures of the drawings. It is intended that the embodiments and figures disclosed herein are to be considered illustrative rather than restrictive.

FIG. 1 is a schematic diagram of the experimental setup for electrophoretic deposition of HAP powder on coronary stents.

FIG. 2 is a schematic flowchart for the production of HAP-coated coronary stents via EPD.

FIG. 3 is a graph illustrating particle size distribution of calcium phosphate particles in a slurry after one week of sedimentation.

FIG. 4 is a micrograph illustrating the surface of a stent after alkali micro-etch treatment.

FIG. 5(a) is a micrograph illustrating the microstructure of a coating prepared under the following EPD conditions: HAP concentration of 0.5 wt %, voltage=2 V, and deposition time=10 seconds.

FIG. 5(b) is a micrograph illustrating the microstructure of a coating prepared under the following EPD conditions: HAP concentration of 0.5 wt %, voltage=2 V, and deposition time=30 seconds.

FIG. 5(c) is a micrograph illustrating the microstructure of a coating prepared under the following EPD conditions: HAP concentration of 0.5 wt %, voltage=5 V, and deposition time=10 seconds.

FIG. 5(d) is a micrograph illustrating the microstructure of a coating prepared under the following EPD conditions: HAP concentration of 0.5 wt %, voltage=5 V. and deposition time=30 seconds.

FIG. 5(e) is a micrograph illustrating the microstructure of a coating prepared under the following EPD conditions: HAP concentration of 1.5 wt %, voltage=2 V, and deposition time=10 seconds.

FIG. 5(f) is a micrograph illustrating the microstructure of a coating prepared under the following EPD conditions: HAP concentration of 1.5 wt %, voltage=2 V, and deposition time=30 seconds.

FIG. 5(g) is a micrograph illustrating the microstructure of a coating prepared under the following EPD conditions: HAP concentration of 1.5 wt %, voltage=5 V, and deposition time=10 seconds.

FIG. 5(h) is a micrograph illustrating the microstructure of a coating prepared under the following EPD conditions: HAP concentration of 1.5 wt %, voltage=5 V, and deposition time=30 seconds.

FIG. 5(i) is a micrograph illustrating the microstructure of a coating prepared under the following EPD conditions: HAP concentration of 2.5 wt %, voltage=5 V, and deposition time=30 seconds.

FIG. 6 are micrographs illustrating at three different magnifications the microstructure of a coating prepared under the following EPD conditions: HAP concentration of 2.5 wt %, voltage=5 V, and deposition time=30 seconds.

FIGS. 7(a) and 7(b) are micrographs illustrating the microstructure of a HAP coating prepared by EPD. The complete uniform coverage of the substrate surface by the use of narrow particle size distribution of HAP powder is shown in 7(a), and as received powder, i.e., a wide particle size distribution of HAP powder is shown in 7(b).

FIGS. 8(a) and 8(b) are micrographs illustrating the behaviour of a HAP coating on an expanded 316L stainless steel stent, without prior surface micro-etching through alkali treatment.

FIGS. 9(a) and 9(b) are micrographs illustrating the behaviour of a HAP coating on an expanded 316L stainless steel stent, with prior surface micro-etching through alkali treatment.

FIGS. 10(a), 10(b) and 10(c) are micrographs illustrating the retention of HAP coatings on an expanded 316L stainless steel stent with surface micro-etched through alkali treatment, showing high-strain regions of the expanded stent.

### DETAILED DESCRIPTION OF THE INVENTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

In the following description, the term “calcium phosphate” is used generically and includes minerals such as HAP, dicalcium phosphate, tricalcium phosphate, tetracalcium phosphate and amorphous or partially amorphous calcium phosphate.

The invention in one embodiment is directed to a process of coating an implantable medical device with calcium phosphate by pretreating a substrate with an alkaline solution, preparing a slurry comprising a desired size range of particles of calcium phosphate, immersing the pretreated substrate in the slurry, and coating the calcium phosphate particles onto the pretreated substrate by electrophoretic deposition. The process results in a thin, uniform, porous calcium phosphate coating that can withstand flexing of the substrate.

The novel coating process is exemplified below with reference to stents, such as cardiovascular stents (e.g. coronary stents). As shown in the examples below, the coating withstands simulated stent expansion procedures. However, the invention has broad application to virtually any type of implantable device with a metallic surface for use in the human or animal body, and particularly to flexible implantable devices. For example, the coatings are also useful in ureteral stenting and catherterisation.

The novel process involves treating the substrate in an alkaline solution to enhance the adhesion of deposited calcium phosphate layer to the substrate. Alkali treatment may be performed by soaking the substrate in a NaOH solution or other suitable alkaline solution, for example. After the alkali treatment, the substrate is rinsed to remove residual alkali material, dried and then heat-treated. Heat treatment could, for example, involve heating at 500° C. for one hour.

As shown in the examples below, alkali treatment positively affects the bonding strength of the calcium phosphate coating. Alkali treatment etches the surface of the substrate and forms sodium chromate. Both results are believed to account for the subsequent improved bonding of the coating to the substrate. Sodium chromate is believed to make a strong bond from one side to the metallic bonds of the substrate, and from the other side to the covalent bonds of the calcium phosphate particles.

The novel process also involves the preparation of a stable colloidal suspension of calcium phosphate particles. The solvent used for the colloidal suspension may be an alcohol, such as ethanol. The slurry may comprise a particular weight percentage range of calcium phosphate, for example ranging from 0.5 to 20 wt %. The colloidal suspension may also comprise calcium phosphate particles in a particular size range. A particular size range of calcium phosphate particles may be obtained by, for example, by gravity sedimentation and/or centrifuge sedimentation. The desired particles may, for example, range in size from 50 nm to 150 nm in diameter. Fine particles sometimes agglomerate but such agglomeration may be eliminated by ultrasonification prior to coating.

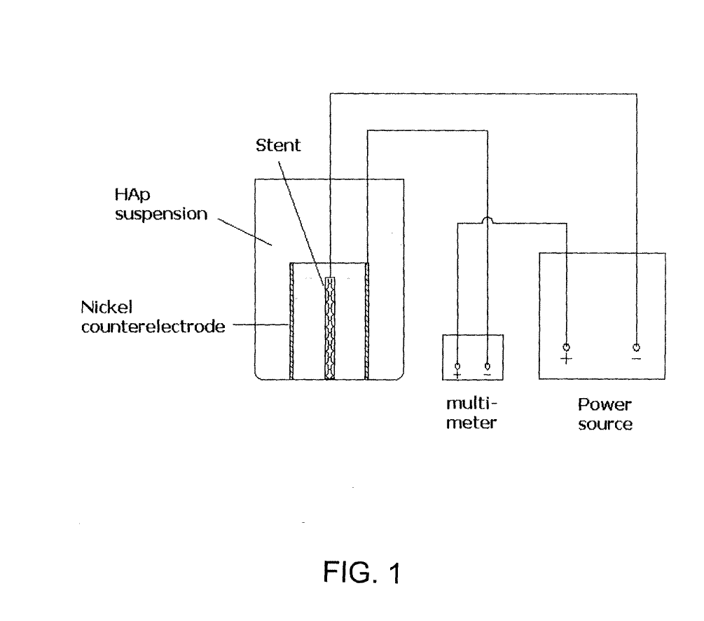

FIG. 1 shows the EPD set-up of one particular embodiment of the present invention. The stent is suspended by a stainless steel wire. The counterelectrode is cylindrically-shaped to provide a uniform distribution of electrical field and is made, for example, from nickel foil. The radial distance between the stent and the counterelectrode is constant. Deposition can be conducted under a range of voltages (e.g. 1 to 5 volts) for a range of times (e.g. 1 to 60 seconds) at room temperature. The coating applied may have a thickness no greater than 1 μm, for example.

After EPD, the coating is dried at room temperature, and then cured. Curing can comprise heating the coated substrate at 500 ° C. for 1 hour, for example. This relatively low curing temperature avoids oxidation damage to certain types of implantable medical devices such as stainless steel stents.

The novel process allows the achievement of optimum coating thickness, coverage uniformity, and maximum coating adhesion. This, in turn, allows the coatings to withstand stresses applied to the substrate, such as, in the case of stents, during and after stent implantation and expansion. Optimum conditions to achieve a coating with a maximum mechanical integrity under applied deformation (e.g. expansion) can be determined by varying substrate parameters such as the stent material, pre-treatment conditions such as the concentration of the alkaline solution, and coating parameters such as coating thickness, particle size, particle concentration, applied voltage, and the deposition duration.

The present invention provides for uniform distribution of calcium phosphate on all outer surfaces of the substrate. For example, all surfaces of a stent, including the wall surface of perforated portions of the stent, can be uniformly coated. Other methods of deposition, such as aerosol-gel, or plasma spraying are not able to provide the same uniform coverage and porous microstructure. Also, in comparison with other methods of depositing calcium phosphate coatings on implantable devices, such as electro-chemical deposition (ECD) technology, the EPD deposits well-developed and well-characterized particles of calcium phosphate onto the substrate.

Further improvement of the functional properties and reliability of the calcium phosphate coatings, depending on the type of implantable medical device, can be achieved through impregnation with polymers, or polymers containing drug, for long-term controlled release. For example, porous calcium phosphate coatings, in particular HAP coatings, can be used as an inorganic scaffold for carrying organic materials, forming a unique organo-ceramic composite. Organic materials may be either co-deposited with the calcium phosphate particles, or impregnated into the coating after calcium phosphate particle deposition.

### EXAMPLES

To demonstrate the feasibility of the novel processing concepts outlined above, the following examples are described below for a stainless steel substrate, in particular, coronary stents. The procedures outlined below can be applied to other implantable medical devices.

### Example 1

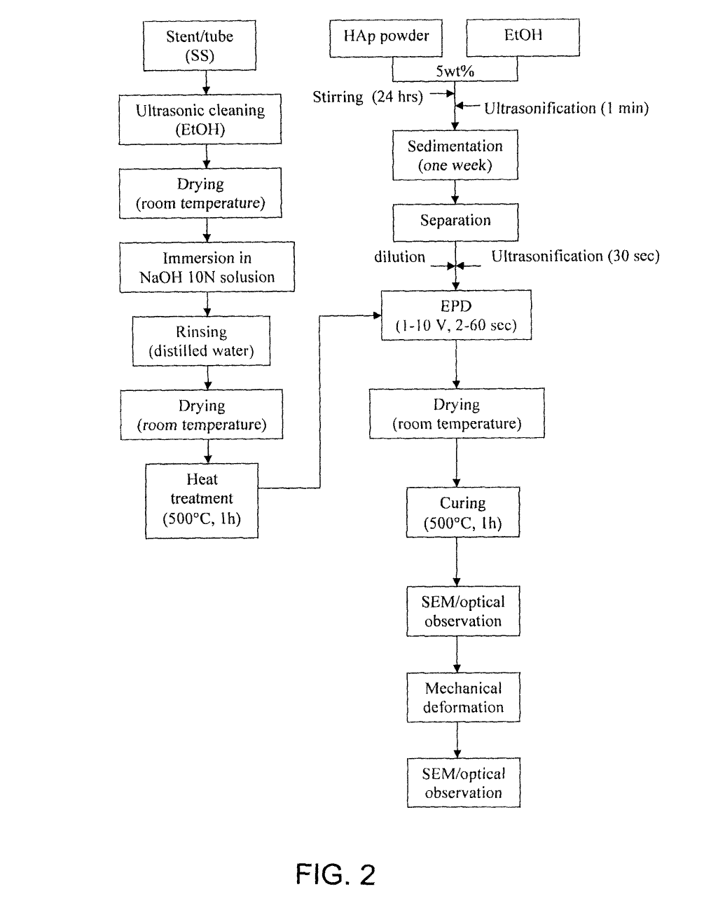

FIG. 2 illustrates the steps taken to coat a stent with HAP according to this example. Electropolished cardiovascular (e.g. coronary) stents made of stainless steel 316L (14 mm length, 1 mm radius) were thoroughly cleaned by immersing in an EtOH ultrasound bath, and vibrated for 5 minutes.

Commercially available HAP powder (Riedel-deHaën) was used as the source of calcium phosphate. For comparative purposes, a number of different HAP powder suspensions were investigated to assess their relative stability. HAP concentration in the slurry was varied from 0.5 to 20 wt %. The solvent used for the suspension preparation was absolute ethanol, mixed with the HAP for 24 hours, and then ultrasonicated for 1 minute to break any agglomerates.

The prepared suspension was allowed to settle and characterized in 24 hour intervals to determine particle size distribution in the different portions of the suspension. Gravitational sedimentation separated the larger particles and agglomerated granules from the fine particles. An upper portion of the suspension containing fine particles was siphoned out by a pipette. It contained particles with an average size of approximately 120 nm. This stable colloidal suspension was found to possess a long shelf life (>1 month). The prepared suspension was then diluted to 10, 30, and 50 vol % of its original concentration to examine the effect of HAP concentration on coating quality.

To eliminate any formed agglomerates, the suspensions were subjected to ultrasonic dispersion for approximately 30 seconds prior to the EPD coating process. HAP colloidal particles, suspended in ethanol, are charged positively. FIG. 1 shows schematically the EPD set-up. The cleaned stent was suspended by a stainless steel wire within a cylindrically-shaped counterelectrode made of nickel foil. The constant radial distance between the stent and the counterelectrode was approximately 1.2 cm. A uniform distribution of electrical field was achieved due to use of the cylindrical counterelectrode. HAP deposition was conducted under conditions of constant voltage at 1 to 5 volts, for the periods of time 1 to 60 seconds, at room temperature. DC current was supplied and controlled by a precise power source. A multimeter measured the change in electrical current change with time, as the deposited HAP layer built up.

After EPD, the coated stents were dried at room temperature, and then cured at 500° C. for 1 hour.

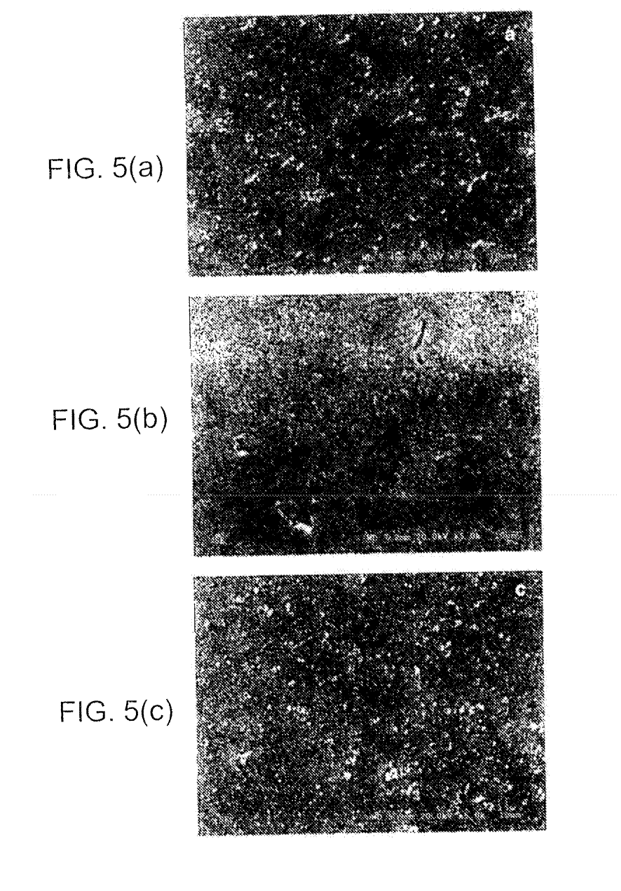

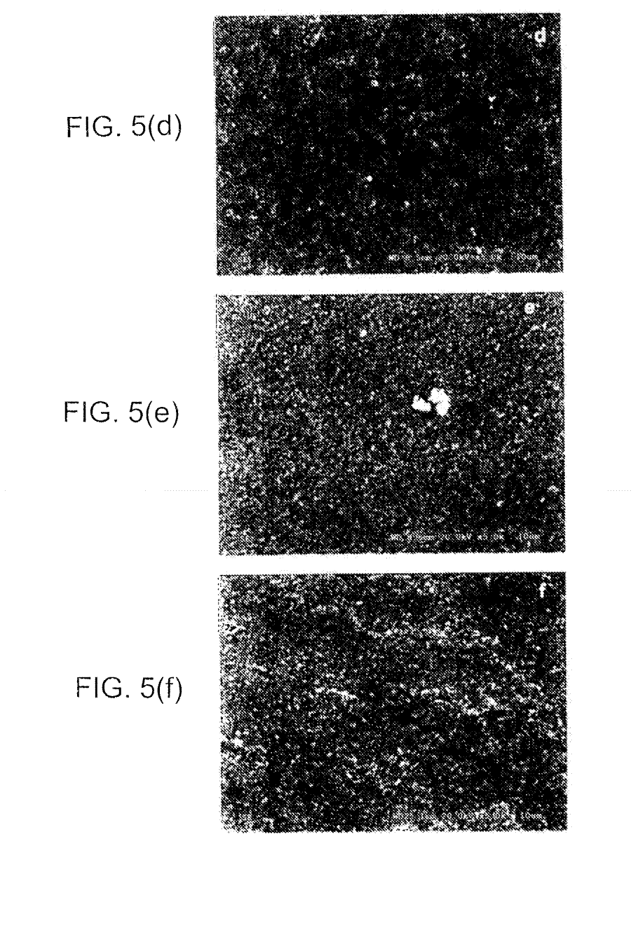

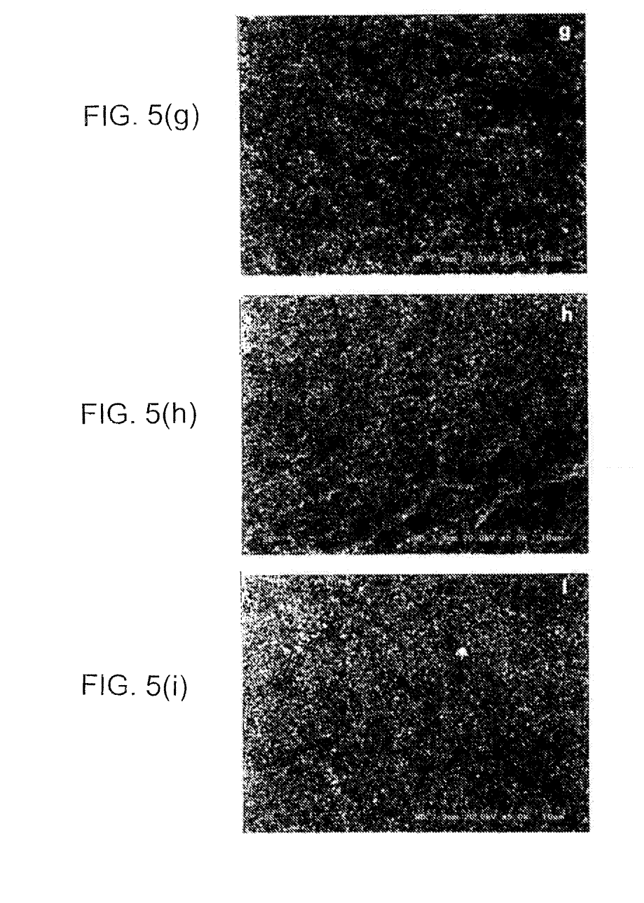

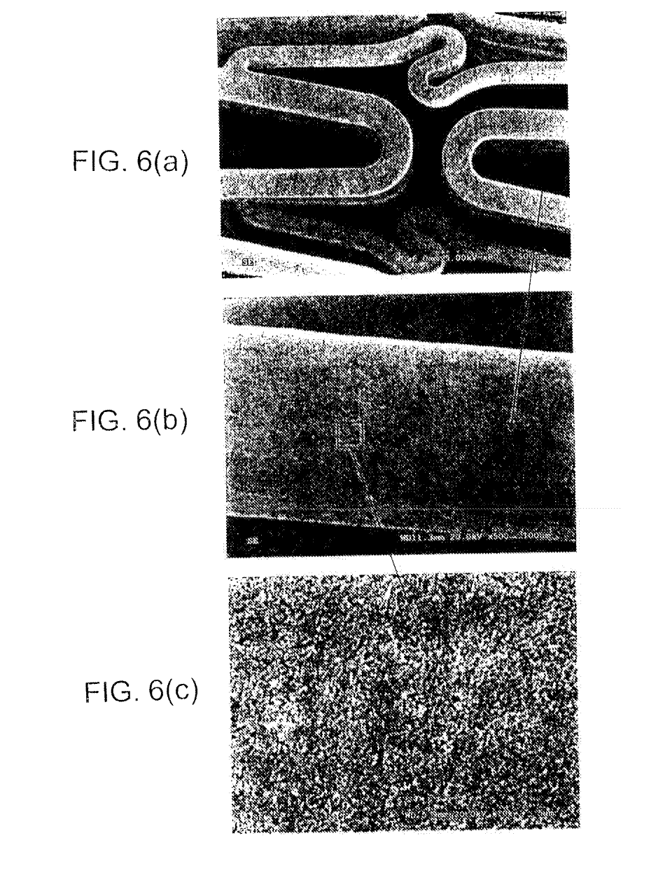

FIGS. 5(a) to (i) and FIG. 6 show the representative appearance and microstructure of the HAP coating prepared under different EPD conditions.

### Example 1a

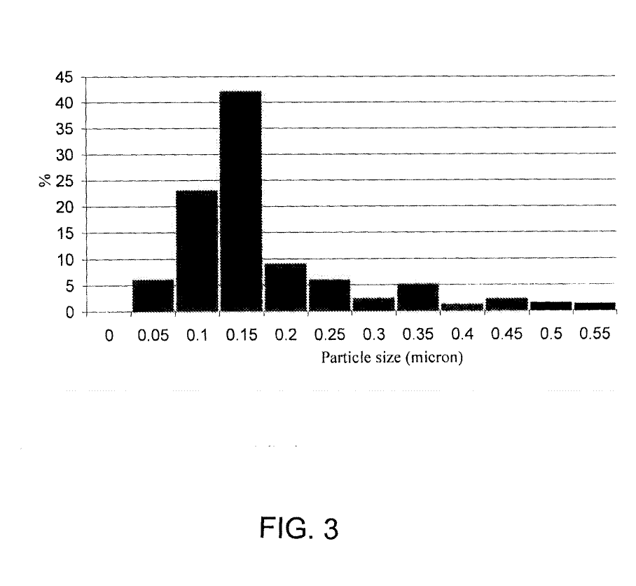

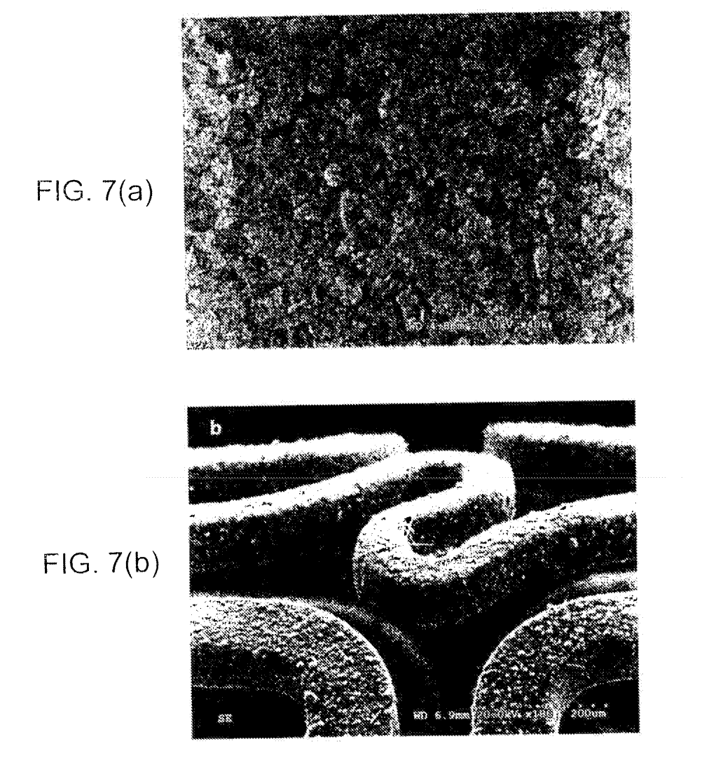

The deposition of HAP was carried out under the following EPD conditions: 2.5 wt % of HAP in the slurry, 5 V voltage, and 30 second deposition time. The HAP in the slurry was in a narrow size distribution, i.e., more than 75% of the particles were in the size range of 50-150 nm. This size distribution is shown in FIG. 3. This size distribution was obtained by siphoning off the top portion of the slurry after a one-week sedimentation process to separate the agglomerates and coarse particles from the finer particles. This separation process may be equally accomplished through sedimentation using centrifuge in less than one hour. The uniform HAP coating which resulted from the use of this narrow size distribution of the particles is shown in FIG. 7a. FIG. 7a has been taken at a very large magnification (40,000×) in order to visualise the fine (approximately 50 nm to 100 nm), uniformly distributed porosity of the HAP coating.

### Example 1b

The deposition of HAP coating was executed similarly as in Example 1a (2.5 wt % of HAP in the slurry, 5 V voltage, and 30 second deposition time), but was carried out by using HAP in the slurry in a broad particle size distribution (from approximately 10 nm to 5 μm). The coarse HAP coating which resulted from the use of this broader range of size distribution of particles is shown in FIG. 7b.

### Example 1c

Stability of the prepared HAP slurry was verified by comparing water and ethanol as the suspension solvent. The sedimentation time of the HAP particles, for a 5 wt % suspension was increased from less than an hour in the water-based suspension to more than a month for the ethanol-based suspension. Although it was possible to prepare stable water-based suspensions of HAP by decreasing pH to a strongly acidic range, water-based suspensions were not pursued to avoid the dissolution of HAP in such an acidic environment.

### Example 1d

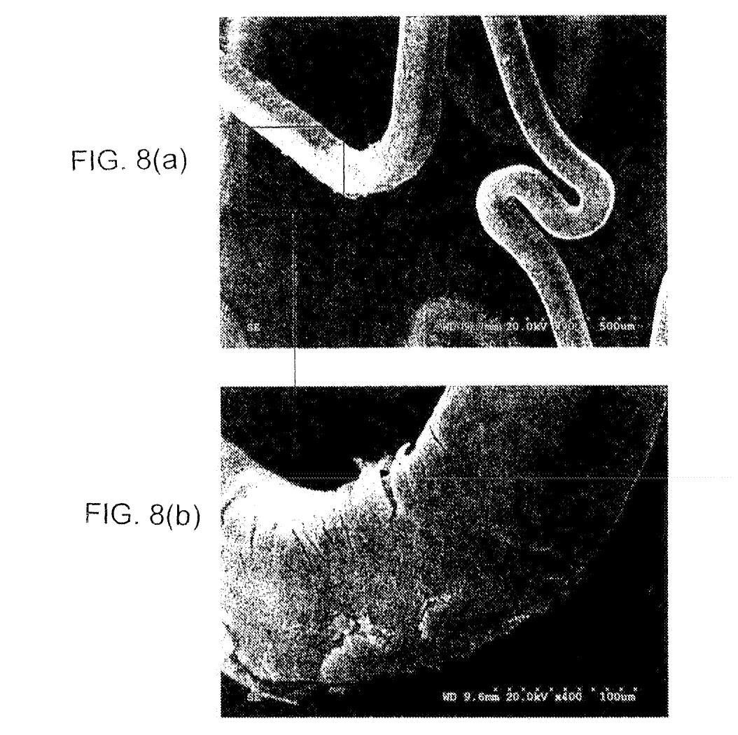

The stent in this example was processed as in Example 1, and then expanded from an initial radius of about 1 mm to a final radius of about 3 mm. The expansion test was performed using Encore™ 26 Inflation Device Kit. The expanded stent was observed under a scanning electron microscope (SEM). FIG. 8 illustrates the results. The HAP coating, processed according to the protocol described in Example 1, separated from the stent surface in areas of significant strain due to stent expansion. The flaked coating allowed the assessment of the coating thickness, which was found to be in the range of 1.0-1.5 μm. The coating was retained in areas experiencing little or no strain.

### Example 1e

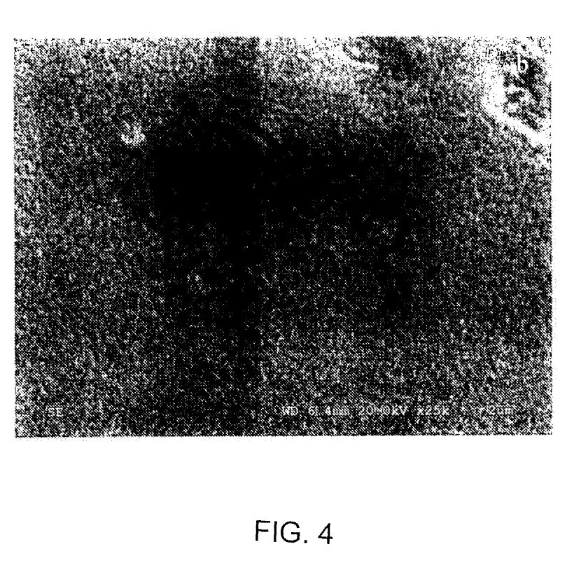

The stent in this example was modified through alkali pretreatment in order to obtain a better adhesion between the coating and the stent. Alkali treatment was performed by soaking the stent in 10 mL of 10N NaOH solution at 60±5° C. for 24 hours. FIG. 4 shows the etched surface of a stent after alkali treatment. After the alkali treatment, the stent was rinsed with distilled water several times, and then dried at room temperature for about 6 hours. The rinsed and dried alkali-treated stent was then heated to 500° C. at a rate of 10° C./min, maintained at that temperature for 1 hour, and then cooled to room temperature at a rate of 1.5° C./min.

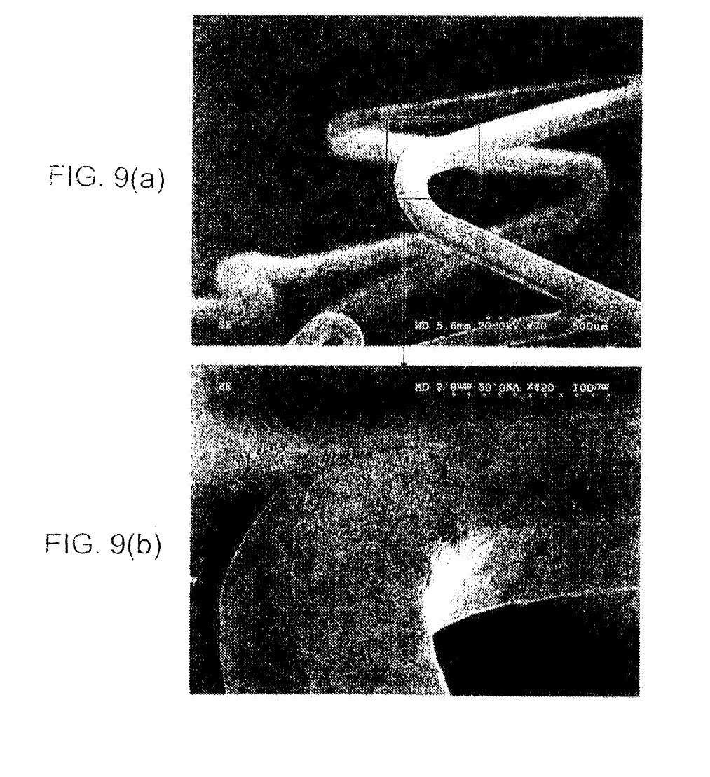

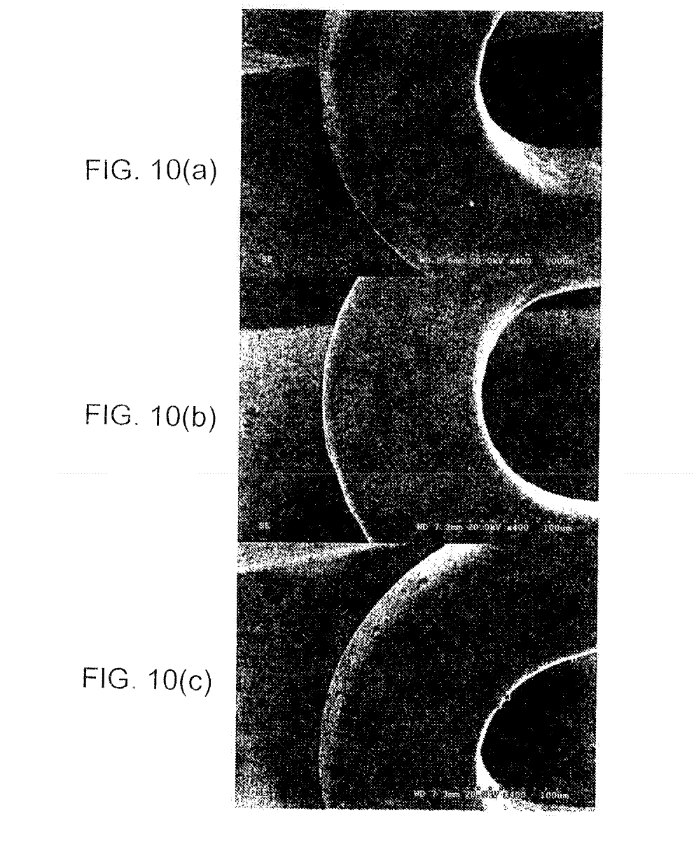

The pretreated stent was coated according to the protocol described in Example 1, and then expanded according to the protocol described in Example 1d. The expanded stent was observed under SEM. FIGS. 9 and 10 illustrate the results. Notably, the HAP coating did not separate from the stent surface, even in areas of significant strain resulting from stent expansion.

1. A process of coating an implantable medical device with a calcium phosphate coating comprising:
(a) treating a metal substrate with an alkaline solution to enhance adhesion of calcium phosphate on the surface of the substrate;
(b) rinsing and heating the alkali-treated substrate and then cooling the substrate to room temperature;
(c) preparing a slurry comprising an organic solvent and a size range of calcium phosphate particles of approximately 50 nm to 150 nm;
(d) immersing the treated substrate in the slurry; and
(e) coating the calcium phosphate particles onto the surface of the treated substrate by electrophoretic deposition.

2. A process according to claim 1 wherein the calcium phosphate particles are obtained by gravity sedimentation or centrifuge sedimentation.

3. A process according to claim 1 wherein the thickness of the coating after performance of step (e) is less than or equal to 1 μm.

4. A process according to claim 1 wherein the substrate is stainless steel.

5. A process according to claim 4 wherein heating the alkali-treated substrate is conducted at 500° C. for about one hour.

6. A process according to claim 1 wherein the alkaline solution comprises sodium hydroxide.

7. A process according to claim 1 wherein the concentration of calcium phosphate particles in the slurry is between 0.5 to 20 wt %.

8. A process according to claim 1 wherein the electrophoretic deposition is conducted at a voltage range of between 1 to 5 volts.

9. A process according to claim 1 wherein the electrophoretic deposition is conducted for between 1 to 60 seconds.

10. A process according to claim 1 wherein the concentration of calcium phosphate particles in the slurry is 2.5 wt % and the electrophoretic deposition is conducted at 5 volts for 30 seconds.

11. A process according to claim 1 further comprising sintering the coated substrate after step (e).

12. A process according to claim 11 wherein the sintering is conducted at approximately 500° C. for one hour.

13. A process according to claim 1 wherein the solvent is an alcohol.

14. A process according to claim 13 wherein the alcohol is ethanol.

15. A process according to claim 1 wherein the calcium phosphate is selected from the group consisting of hydroxyapatite, dicalcium phosphate, tricalcium phosphate and tetracalcium phosphate.

16. A process according to claim 1 wherein the calcium phosphate is hydroxyapatite.

17. A process according to claim 1 wherein the substrate is flexible.

18. A process according to claim 1 wherein the substrate is a stent.

19. A process according to claim 18 wherein the stent is a cardiovascular stent.

20. An implantable medical device made by
(a) treating a metal substrate with an alkaline solution to enhance adhesion of calcium phosphate on the surface of the substrate
(b) rinsing and heating the alkali-treated substrate and then cooling the substrate to room temperature;
(c) preparing a slurry comprising an organic solvent and a size range of calcium phosphate particles of approximately 50 nm to 150 nm;
(d) immersing the treated substrate in the slurry; and
(e) coating the calcium phosphate particles onto the treated substrate by electrophoretic deposition.

21. An implantable medical device according to claim 20 wherein the substrate is flexible.

22. An implantable medical device according to claim 21 wherein the substrate is a stent.

23. An implantable medical device according to claim 22 wherein the stent is a cardiovascular stent.

24. A process according to claim 16, wherein more than 75% of the hydroxyapatite particles have the size range of 50-150 nm.

25. An implantable medical device according to claim 20, wherein the calcium phosphate is hydroxyapatite.

26. An implantable medical device according to claim 25, wherein more than 75% of the hydroxyapatite particles have the size range of 50-150 nm.
